(19) United States
(12) Patent Application Publication
(10) Pub. No.: US 2008/0241091 A1
(43) Pub. Date: Oct. 2, 2008

(54) Air Freshener

(75) Inventors: Thomas McGee, Nyack, NY (US); Christopher Owen Louis Smith, Sugar Loaf, NY (US)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 11/572,596

(22) PCT Filed: Jul. 15, 2005
(86) PCT No.: PCT/CH05/00413

§ 371 Date: Feb. 14, 2007

Related U.S. Application Data
(60) Provisional application No. 60/591,275, filed on Jul. 27, 2004.

(51) Int. Cl. A61L 9/01; A01P 3/00

(52) U.S. Cl. 424/76.8

(57) ABSTRACT

Means of disseminating simultaneously into an atmosphere a volatile antifungal fragrance and a non-antifungal fragrance, the two fragrances being disseminated from two different sources such that the non-antifungal fragrance is more olfactively perceptible than the antifungal fragrance. Typically the non-antifungal fragrance is present in the packaging of the antifungal fragrance.

This invention relates to a system for simultaneously providing a pleasant fragrance and prevention of fungal growth.

Mould growth in the home is a recognized consumer problem, causing discoloration of walls and wallpaper and unpleasant odors in clothing and footwear. It is also recognized as a health hazard as some moulds generate spores that can cause allergic reactions in humans.

Antifungal products in the form of sprays are effective but cannot be used on all surfaces as they can cause damage and discoloration. In an effort to control mould growth without the disadvantages of liquid sprays, the antifungal properties of fragrance compositions in the vapour phase have been explored, but the levels of active ingredients required for controlling mould growth restricts the ability of the perfumer to create a pleasant smell. A recent attempt to achieve simultaneously the objects of preventing the growth of fungi on substrates and imparting a pleasant odour has involved the use of an anti-mould fragrance composed of at least two fragrance components selected from the group consisting of certain selected cyclic aldehydes, cyclic alcohols, branched or unbranched linear aldehydes, branched or unbranched linear alcohols, phenols and lactones. Although the hedonic qualities are improved, the need to use a high level of antifungal active components still restricts the perfumer's ability to create different fragrance notes.

It has now been discovered that it is possible to provide effective antifungal activity with a wider range of desirable odours by utilizing a particular means. The invention therefore provides means of disseminating simultaneously into an atmosphere a volatile antifungal fragrance and a non-antifungal fragrance, the two fragrances being disseminated from two different sources such that the non-antifungal fragrance is more olfactively perceptible than the antifungal fragrance.

The invention further provides a method of disseminating simultaneously in an atmosphere an antifungal fragrance and a non-antifungal fragrance, comprising providing the antifungal fragrance and the non-antifungal fragrance in separate sources selected such that the dissemination rate of the antifungal fragrance is sufficient to provide a desired antifungal effect and the dissemination rate of the non-antifungal fragrance is such that it is more olfactively perceptible than the antifungal fragrance.

By “means” is meant any means capable of disseminating two fragrances simultaneously into an atmosphere. The use in this specification of the term “means” includes delivery systems. There is a wide variety of possibilities allying within the scope of the invention, and specific preferred embodiments are further discussed hereinunder.

Antifungal fragrances are those that provide an antifungal effect. The existence of this effect in any given fragrance may be determined by placing various amounts of the fragrance in a gas-tight container with remotely-placed Petri dishes containing mould inoculum. Such fragrances should be capable of diffusing into the vapour phase to give a head space concentration of antifungal fragrant ingredients above their minimum inhibitory concentration.

The antifungal fragrance compositions must be effective against all common household fungi such as Aspergillus, Cladosporium, Penicillium, Phoma, and Aureobasidium.

Examples of suitable antifungal fragrances include those mentioned in U.S. application 60/547,120, the disclosures of which are incorporated herein by reference. It is possible to use more than one such antifungal fragrance. Examples of suitable antifungal fragrances include:

a) cyclic aldehydes, for example, 2-methyl-3-phenyl-2-propenal, 2-phenyl-propanal, 4-methyl-benzaldehyde, 2-phenyl-ethanal, 3-phenyl-propanal, 4-methyl-phenyl acetaldehyde, 4-methoxy-benzaldehyde, 1-carboxaldehyde-2,4-dimethyl-cyclohex-3-en, 3-(4-methoxyphenyl)-2-methyl-propanal, 1,3-benzodioxole-5-carboxaldehyde, 3-methyl-5-phenyl-pentanal, 1-carboxaldehyde-2,4,6-trimethyl-cyclohex-3-en, alpha-methyl-1,3-benzodioxole-5-propanal;
b) cyclic alcohols, for example, 3-phenyl-2-propen-1-ol, 4-(1-methylethyl)-benzene methanol, 2-phenyl-ethanol, 3-phenyl-propanol, 3-(4-methyl-3-cyclohexenyl)-butanol, 2-methyl-4-phenyl-butan-2-ol, 2,2-dimethyl-3-(3-methyl phenyl)-propanol, 3-methyl-5-phenyl-pentanol, 2-methyl-5-phenyl-pentanol;
c) branched or unbranched linear aldehydes, for example, 3,7-dimethyl-octa-2,6-dien-1-al, 2,4-nonadienal;
d) branched or unbranched linear alcohols, for example, from 10-undecenol, 1-nonanol, (e)-3,7-dimethyl-octa-3,6-dienol, (z)-3,7-dimethyl-octa-3,6-dienol, 3,7-dimethyl-6-octen-1-ol, 9-decenol, 2,6-nonadienol;
e) phenols, for example, carvacrol, dihydro eugenol, eugenol, isoeugenol, thymol; and
f) lactones, for example, 5-hexyl-furan-2(3h)-one, dihydro-5-pentyl-2(3h)-furanone, 4-methyl-5-pentyl-dihydro-2(3h)-furan-2-one, 8-methyl-1-oxaspiro[4,5]-decan-2-one.

In a preferred embodiment of this invention, at least two of these antifungal fragrances are present.

The antifungal fragrance is present to the extent of at least 50% by weight, and more preferably at least 70% by weight of the total fragrance present in the means. It may be formulated neat as a liquid contained in a suitable package, or it may be formulated with any convenient delivery vehicle known to be useful for delivering fragrance compositions. For example, the fragrance may be incorporated into a gel, encapsulated in granules or absorbed on to porous bodies to form powders. Preferred delivery vehicles are described in more detail hereinunder.

The non-antifungal fragrance is one that provides a pleasant smell combining with or masking the odor of the antifungal fragrance. It can selected from one or more compounds, for example, natural products such as extracts, essential oils, absolutes, resinoids, resins, concretes etc., and also synthetic materials such as hydrocarbons, alcohols, aldehydes, ketones, ethers, acids, esters, acetals, ketals, nitrites, etc., including saturated and unsaturated compounds, aliphatic, carbocyclic, and heterocyclic compounds. The molecular weights range from around 90 to 320. Such fragrance materials are mentioned, for example, in S. Arctander, Perfume and Flavor Chemicals (Montclair, N.J., 1969), in S. Arctander, perfume and Flavor Materials of Natural Origin (Elizabeth, N.J., 1960) and in “Flavor and Fragrance Materials—1991”, Allured Publishing Co. Wheaton, Ill. USA.

The non-antifungal fragrance comprises at least 20%, preferably greater than 30% of fragrance components, as hereinafter described, having a vapour pressure at 25° C. of greater than 0.01 mm Hg. More preferably, the vapour pressure should also be less than 10 mm Hg. Vapour pressure may calculated from the structure of the molecule using one of the commercially available software programs, such as ACD Software, ACD/Boiling Point calculator version 4.0.

In addition, the non-antifungal fragrance components should have a low perception threshold, preferably below 500 nanograms per liter. The lower the perception threshold of an aroma chemical, the lower the quantity that is required to smell it. A variety of techniques are available to determine the perception threshold. See, for example, Neuener-Jehle and Etzweiler in Art Science and Technology, (ed. Lamparski and Muller, Elsevier, 1991), 153-212.

Typical non-antifungal fragrances suitable for use in this invention include: acetophenone, alcohol c6, aldehyde c7, aldehyde c8, aldehyde c12 mna, allyl amyl glycolate, amyl acetate, anisyl acetate, aubepine anethol, benzaldehyde, butyl butyrate, carvone laevo, cis-3 hexenyl formate, cis-3-hexenol, corps cassis, cyclohexyl acetate, dihydro myrcenol, dihydro anethole, dihydro rose oxide, ethyl caproate, ethyl-2-methyl butyrate, eucalyptol, florhydral, folione heliotropine, hexyl acetate, hexyl propionate, indole, fructone, ethyl phenyl acetate isobutyl acetate, limonene, d-, limonene laevo, lime oxide, manzanate, melonal, methyl amyl ketone, methyl anthranilate, methyl heptenone, methyl octalactone, methyl octyl acetaldehyde, methyl phenyl acetate, methyl amyl ketone, methyl hexyl ketone, methyl phenyl ethyl oxide, myrcene super, nonenol, cis-6, ocimene, para cresyl acetate, para cresyl isobutyrate, para tolyl aldehyde, phenyl ethyl alcohol, phenyl ethyl formate, phenyl acetaldehyde 85, phenyl propyl aldehyde prenyl acetate, octenolid, valspice, yara yara.

The antifungal fragrance may be incorporated in a delivery vehicle in amounts that may depend on the nature of the delivery vehicle, and the nature of the atmosphere to be treated, for example its volume and the degree of humidity to be encountered. These systems are designed to maintain the fragrance concentration above the minimum inhibitory concentration for moulds (MIC) over the useful life of the system. Preferred (but not limiting) examples of suitable delivery systems include the following:
(a) The antifungal fragrance is dispensed via a diffusion medium in the form of a gel. The most preferred gels are those that can contain a fragrance load in excess of 50% (w/w) such that the level of the specified antifungal fragrance composition in the vapor phase is always greater than MIC over the useful life of the product.
(b) The antifungal fragrance is in a liquid form, and it is diffused via a porous wick-type system, optionally including a heater and/or a fan to promote fragrance evaporation and diffusion into the surrounding environment such that the level of the specified antifungal fragrance composition in the vapor phase is always greater than MIC over the useful life of the product.
(c) The antifungal fragrance is dispensed via a nebulization system which may include a fan to provide a level of the specified antifungal fragrance composition greater than the MIC over the useful life of the product.
(d) The antifungal fragrance is diffused from a porous solid matrix such that the level of the specified fragrance composition such that the level of the specified antifungal fragrance composition in the vapor phase is always greater than MIC over the useful life of the product. The solid matrix is preferably in the form of a powder, which may be composed of porous or absorbent bodies that take up the anti-fungal fragrance composition by absorption or by impregnation. Alternatively, the powder may comprise granules that encapsulate the anti-fungal fragrance composition, made, for example, by spray-drying and coacervation. All of these are well known to the art and their application to the present invention is straightforward. The powders preferably are capable of having a composition load in excess of 20%, preferably 30% by weight. They can be contained any suitable container that allows fragrance to diffuse.

The non-antifungal fragrance is delivered to the atmosphere in any suitable manner, provided that it is delivered independently of the antifungal fragrance. It is possible simply to use one of the delivery systems hereinabove mentioned. For example, a delivery system may have two reservoirs and two associated wicks, each delivering one or other of the antifungal or non-antifungal fragrance. However, in a preferred embodiment the non-antifungal fragrance is incorporated into a solid plastics material. This may be achieved by known means, for example, by impregnating a porous plastic. The plastics material may be any suitable part, for example, it may be the material of the container containing a liquid antifungal fragrance.

Suitable plastics materials within which a fragrance may be incorporated are well known in the art. Examples include high- and low-density polyethylene, polypropylene, polyvinyl chloride, polyether block amides and copolymers. Techniques for incorporating fragrances into plastics are well known, and include (but are not limited to) U.S. Pat. Nos. 3,688,985, 3,994,439, 4,095,031 and 4,552,693, the disclosures of which and others teaching similar techniques are incorporated herein by reference.

In another embodiment, the non-antifungal fragrance incorporated into the plastic has water-soluble fragrance components comprising no more than 20% by weight, and more preferably no more than 15% by weight of the total composition. Higher levels make the plastic feel undesirably wet to the touch.

The fragrances may also comprise volatile solvents. These may be selected from any known to the art, and non-limiting examples include ethers; straight or branched chain alcohols and diols; volatile silicones; dipropylene glycol, triethyl citrate, ethanol, isopropanol, diethyleneglycol monoethyl ether, dipropylene glycol, diethyl phthalate, triethyl citrate, isopropyl myristate, etc., hydrocarbon solvents such as Isopar™ or other known solvents that have previously been used to dispense volatile actives from substrates. These solvents in general have a molecular weight between 20 and 400. They are selected specifically for each volatile liquid to achieve the performance and safety, (e.g. VOC and flash point) specified. With reference to the previous paragraph, it is preferred that the quantities of water-soluble or -miscible polar solvents such as monohydric alcohols, dipropylene glycol, and 3-methyl 3-methoxy butanol be minimized or even completely excluded from the non-antifungal fragrance composition.

The invention is now further described with reference to the following non-limiting example, which describes a preferred embodiment.

### EXAMPLE

10 g samples of the following crosslinked, polymeric gel composition described in WO03/020412 were prepared and placed in Petri dishes:

| | % (w/w) |
|---|---|
| Lithene ™ N4-9000-10MA* | 17.0 |
| Steol CS-460** | 5.0 |
| Jeffamine D-400*** | 2.5 |
| 1% Dye solution | 1.0 |
| Antifungal fragrance | 74.5 |

*liquid polybutadiene polymer, ex Synthomer Ltd.
**liquid sodium laureth sulfate, ex Stepan Products
***polyoxypropylenediamine crosslinking agent, ex Huntsman Chemicals

The antifungal fragrance formulation is as described in Example 2 of U.S. application 60/547,120 and contains 67% cyclic aldehyde, 7% linear aldehydes, 5% linear alcohols active components and 21% non-active fragrance materials.

The following 15% (w/w) fragrances were incorporated into a polyether block amide plastic, Pebax™ 25533 SA01 ex Atofina Chemicals, Inc., Philadelphia, Pa. by adding the fragrance to the plastic beads and tumbling for 1 hour.

| | % (W/W) | | | | |
|---|---|---|---|---|---|
| Fragrance | A | B | C | D | E |
| nerolidol synthetic | 7.5 | 5.0 | 5 | 5 | — |
| cedrol crystals | 7.5 | 5 | 5 | 5 | — |
| cetone v | 15.5 | 7.5 | 5 | 5 | 5 |
| phenyl ethyl benzoate | 11.3 | 6.5 | 5 | 5 | 5 |
| hydroxy citronellal dimethyl acetal | 20 | 20 | 10 | — | 8 |
| maltol iso butyrate | 2.7 | 20 | 20 | 20 | 20 |
| isoeugenol acetate | 2.3 | 20 | 10 | 10 | |
| jasmine lactone gamma | 4.0 | 19 | 10 | 10 | |
| heliotropine | 8.7 | — | 10 | 10 | — |
| anisyl acetate | 47.3 | — | — | — | 10 |
| manzanate | 0.3 | — | — | 10 | 10 |
| dihydro myrcenol | 4.8 | — | — | | 10 |
| amyl acetate | — | — | — | 10 | 20 |
| isopropyl myristate | 1.0 | 10 | 10 | 10 | 2 |

% (w/w) of non-antifungal fragrance with vapour
pressure greater than 0.01 mm Hg at 25 C.

| | A | B | C | D | E |
|---|---|---|---|---|---|
| Press >0.01 | 0 | 10 | 20 | 30 | 30 |

10 gram of the respective fragranced Pebax™ plastic was placed in glass jar.

A Petri dish with the antifungal fragrance and one with the fragranced plastic were placed side by side in a smelling booth and left for 1 hour. A panel of 5 people was asked to rate on a 5 point scale how well the odor of the antifungal fragrance had been covered (1=no coverage; 5 complete coverage). The results were:

| Fragrance | |
|---|---|
| A | 1.0 |
| B | 1.4 |
| C | 3.7 |
| D | 4.4 |
| E | 4.8 |

Good coverage required at least 20% of fragrance materials having a vapor pressure of around 0.01 mm Hg at 25° C. or greater.

1. Means of disseminating simultaneously into an atmosphere a volatile antifungal fragrance and a non-antifungal fragrance, the two fragrances being disseminated from two different sources such that the non-antifungal fragrance is more olfactively perceptible than the antifungal fragrance.

2. Means according to claim 1, in which the antifungal fragrance is present to the extent of at least 20%, and more preferably at least 50%, by weight of the total fragrance present in the means.

3. Means according to claim 1 which the non-antifungal fragrance is composed of at least 20%, preferably greater than 40%, of fragrance components with a vapour pressure greater than 0.01 mm Hg and preferably also less than 10 mm Hg,

4. Means according to claim 1 in which the antifungal and non-antifungal fragrances are contained in a single unit and are separated.

5. Means according to claim 1 in which the non-antifungal fragrance is incorporated into the packaging containing the antifungal fragrance.

6. Means according to claim 1 in which the non-antifungal fragrance is incorporated into plastic which can be attached to or placed close to the pack containing the antifungal fragrance.

7. Method of disseminating simultaneously in an atmosphere an antifungal fragrance and a non-antifungal fragrance, comprising providing the antifungal fragrance and the non-antifungal fragrance in separate sources selected such that the dissemination rate of the antifungal fragrance is sufficient to provide a desired antifungal effect and the dissemination rate of the non-antifungal fragrance is such that it is more olfactively perceptible than the antifungal fragrance.
